(19) United States
(12) Patent Application Publication
(10) Pub. No.: US 2002/0009486 A1
(43) Pub. Date: Jan. 24, 2002

(54) THERAPEUTIC AGENT DELIVERY INCORPORATING REFLECTIVE OPTICAL FILM

(75) Inventor: Kristin J. Godbey, St. Paul, MN (US)

Correspondence Address: Attention: Charles L. Dennis II, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/449,661

(22) Filed: Nov. 30, 1999

(51) Int. Cl. A61F 13/00

(52) U.S. Cl. 424/449

(57) ABSTRACT

A therapeutic agent delivery device can be applied onto a host in order to therapeutically administer a therapeutically active agent to the host, wherein the device incorporates a reflective optical film. Use of reflective optical films in patch devices allows a wide variety of visual elements to be incorporated into these devices without relying upon any colorants, dyes, inks, toners, or other materials. These visual effects include logos, high reflectivity, iridescence, graphics, printed information, surface microstructure, bar codes, color, designs, and/or the like. The present invention also improves the barrier qualities of patch drug devices, prolonging shelf life. This enhanced barrier protection is desirable for all patch therapeutic agent delivery devices, but is particularly beneficial for devices containing fluid reservoirs in which the therapeutically active agent(s) might be stored.

### FIELD OF THE INVENTION

This invention relates to a therapeutic agent delivery device of the type that is attached to a host at an administration site in order to therapeutically deliver a drug, skin wrinkle treatment agent, vitamin, aromatherapy agent, cosmeceutical, nutriceutical, or other therapeutically active agent topically, transdermally, transmucosally, or via other transtissue delivery mechanisms. More specifically, this invention relates to such devices that comprise protective backings and that may optionally comprise a release liner on which the device is releasably stored prior to use, wherein at least one of the backing and/or the release liner incorporates one or more reflective optical films.

### BACKGROUND OF THE INVENTION

Delivery of therapeutically active agents via devices (“patch devices”) that are attached to and worn by a host, whether human or animal or plant, is an increasingly important, non-invasive method of administering therapeutic agents. In practice, a device containing the agent or agents to be administered is placed onto a tissue of a host. The agent, which is releasably stored in a repository of the device, is then caused via diffusional mechanisms or the like to be delivered to the host in furtherance of the desired therapeutic treatment. Such delivery can be used for topical, transdermal, transmucosal, or other transtissue delivery of the agent and to therapeutically treat local or systemic medical conditions. Patch devices can be used for pharmacological treatments, cosmetic treatments, nutriceutical treatments, and/or the like.

A typical patch structure includes a suitable repository that releasably stores an appropriate dosage of one or more therapeutically active agents. The repository layer may be a solid, semi-solid, gel, or liquid. For example, one kind of repository is a liquid in which the therapeutically active agent is dissolved, dispersed, or otherwise distributed. Such liquid repositories are typically stored within a sealed pouch structure incorporated into the patch device. In other patch devices, the repository may be a solid matrix comprising a pressure sensitive adhesive in which the therapeutically active agent is distributed. These and other repository structures are well known and are described, for example, in U.S. Pat. Nos. 5,780,045, 5,851,549, 5,372,819, 5,908,637 and 5,702,720.

In a representative construction, the repository is sandwiched between a protective backing and a release liner. Other layers, such as membrane layers, adhesive layers, barrier layers, or the like, can be incorporated into the device as well. At the time of use, the release liner is easily removed, exposing a tacky surface that allows the patch to be attached to the host. Once the patch is attached to the host, the therapeutic agent leaves the repository and diff-uses and/or otherwise penetrates into the host, or is topically active, in accordance with the desired therapeutic treatment.

In addition to the therapeutically active agent, the repository may further include one or more other components, such as penetration enhancers that help control the rate at which the drug is administered to the host. Most commonly, penetration enhancers are used to increase the rate of drug delivery inasmuch as the skin or mucosa of a host can be an effective barrier against migration of the drug into the host in therapeutically effective amounts. Other optional ingredients that may be incorporated into the repository include solubilizers, fillers, fragrances, flavorings, stabilizers, and the like.

An optional backing may protect the repository and the components contained in the repository, including the therapeutically active agent, from the environment. The backing also prevents loss of ingredients of the device to the environment. Protective backings may be made from a wide variety of materials conventionally used as backing materials in the medical field, including polymeric films and the like.

It is important to ensure the long-term stability of the desired drug formulation when in contact with the backing and release liner. The patch properties could be adversely affected if colorants, dyes, inks, toner, or other materials were to be used to impart color, graphics, printed information, bar codes, logos, surface microstructure, or other visual effects onto a patch device. For instance, some kinds of colorants, dyes, inks, toners, or the like could leach out of the device and be carried with the drug and penetration enhancer to the host. Some of these materials also might tend to interact with one or more device ingredients, perhaps causing accelerated decay of the drug or the penetration enhancer. Some of these materials, such as titanium dioxide which might be added to a formulation for opacity or for white color, might tend to absorb or adsorb oily ingredients (e.g., fatty acid esters commonly used as penetration enhancers) and thereby prevent the absorbed or adsorbed substances from performing their desired function.

Yet, it would nonetheless be desirable to be able to provide logos, graphics, printed information, bar codes, color, designs, surface microstructure, and/or other visual effects, onto patch devices. It would be extremely desirable to be able to achieve this using an approach that is inert with respect to the functional ingredients of the devices.

It also is generally desirable that patch therapeutic agent delivery devices have as long a shelf life as possible so that distributors, merchants, medical professionals, pharmacists, and/or users can keep a reasonable supply of such devices on hand without worrying that the viability of the devices will be reduced, or even lost, upon storage. Shelf life can depend upon a number of factors. One such factor is the barrier quality of the iced protective backing, packaging, and/or release liner that might be used in a particular device. If the barrier quality is too low, formulation ingredients in the device can leach out, leak out, volatilize, dry up, or otherwise be lost, thus shortening shelf life. Shelf life is particularly a concern with patch devices in which the drug repository is a fluid reservoir in that such reservoirs may have a tendency to dry up, harden, or volatilize over time if not adequately protected. Accordingly, it would be desirable to improve the barrier qualities of a patch drug device in order to increase its shelf life.

### SUMMARY OF THE INVENTION

The present invention provides an approach in which a wide variety of visual elements can be incorporated into patch devices without relying upon any colorants, dyes, inks, toners, or other materials. These visual effects include logos, high reflectivity, iridescence, graphics, printed information, surface microstructure, bar codes, color, designs, and/or the like. The present invention also improves the barrier qualities of patch drug devices, prolonging shelf life. This enhanced barrier protection is desirable for all patch therapeutic agent delivery devices, but is particularly beneficial for devices containing fluid reservoirs in which the therapeutically active agent(s) might be stored.

The present invention achieves these advantages through the use of reflective optical films. Reflective optical films are easily incorporated into patch devices in a variety of ways, particularly as the backing, release liner (if any), or both of these. Advantageously, the visual effects demonstrated by reflective optical films are a permanent characteristic of these films and cannot be removed either accidentally or purposely. This allows the visual effects to be used for permanent identification of a patch device during manufacture, storage, and use. It also offers proof of authenticity.

Of course, visual information could still be printed onto these films using conventional printing techniques, but this could introduce undesirable components into the device. Thus, although possible, the use of conventional printing is not necessary and may not even be desirable.

In one aspect, the present invention relates to a therapeutic agent delivery device that can be applied onto a host in order to therapeutically administer a therapeutically active agent to the host, wherein the device incorporates a reflective optical film.

In another aspect, the present invention relates to a therapeutic agent delivery device that can be applied onto a host in order to therapeutically administer a therapeutically active agent to the host. The device includes a repository comprising a releasably stored dosage of the therapeutically active agent. A backing protects the repository, wherein the backing comprises a reflective optical film.

In another aspect, the present invention relates to a therapeutic agent delivery device assembly. The assembly includes a therapeutic agent delivery device that can be applied onto a host in order to therapeutically administer a therapeutically active agent to the host. The device includes a repository comprising a releasably stored dosage of the therapeutically active agent. The device also comprises a backing protecting the repository. The assembly further includes a release liner on which the therapeutic agent delivery device is releasably stored. The release liner incorporates a reflective optical film.

In another aspect, the present invention relates to a method of making a therapeutic agent delivery device. The method involves forming a drug repository that stores a dosage of a therapeutically active agent. The repository is positioned in a therapeutic agent delivery device assembly, said assembly comprising a reflective optical film.

In another aspect, the present invention relates to a method of therapeutically administering a therapeutically active agent. An assembly is provided that includes a patch therapeutic agent delivery device releasably supported upon a release liner. The assembly comprises a reflective optical film. The patch drug device comprises the therapeutically active agent releasably stored in a repository. The patch therapeutic agent delivery device is removed from the release liner and is then adhered to a host in a manner effective to cause the therapeutically active agent to be therapeutically administered to the host.

### BRIEF DESCRIPTION OF THE DRAWINGS

The above mentioned and other advantages of the present invention, and the manner of attaining them, will become more apparent, and the invention itself will be better understood, by reference to the following description of the embodiments of the invention taken in conjunction with the accompanying drawings, wherein:

FIG. 1 is top view of a patch assembly of the present invention.

FIG. 2 is a cross-sectional view of the patch assembly taken along line 2-2 of FIG. 1.

FIG. 3 is a cross-sectional view of an alternative embodiment in which the active ingredient is releasably stored in a liquid reservoir.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

A reflective optical film, as used herein refers to films comprising a structure and/or composition that optically interact with incident light to produce visual effects, including color, reflection, polarization, iridescence, and the like. In one preferred embodiment, reflective optical films of the present invention comprise a stack of film layers, typically co-extruded and including a plurality of one kind of polymer layer interleaved with one or more other layers comprising the same and/or different polymer(s). Optical interaction among the layers produces visual effects. In the aggregate, such a multilayer optical film can include from tens to thousands of layers. Visual patterns are easily created on the surfaces of these films by using embossing, flame impingement, light e-beam treatment, ultraviolet irradiation, laser ablation, or other suitable techniques to disrupt the film layers upon which the patterns are to be formed. This allows lettering, logos, graphics, bar codes, surface microstructure, or any other kind of optically viewable pattern to be formed on the surface. In some instances, the entire surface of a reflective optical film can be treated, such as by flame impingement or by microstructuring, in order to alter the basic color or reflective characteristics. This overall treatment can be used to create surfaces demonstrating soft opalescence or rippling color change effects that can be very aesthetically pleasing.

Multilayer optical films are highly effective barriers. They are highly impermeable to water, water vapor, oxygen, and the like, and can be extremely inert with respect to formulation components used in patch therapeutic agent delivery devices. For instance, these films exhibit a lower moisture vapor transmission rate than solid, single layer polyester films of the same thickness. Thus, multilayer optical films are advantageously used as packaging, release liners, protective backings, fluid reservoir pouches, and/or the like for patch devices. When incorporated into a release liner, low adhesion coatings comprising silicone polymers, fluoropolymers, or the like, can be coated onto one or both surfaces of the film to provide release capabilities if needed. When used as a backing or as a reservoir pouch, it may be desirable to coat one or both surfaces of the film with a coating comprising a heat sealable material such as ethylene vinyl acetate, polyethylene, and/or the like.

Multilayer optical films tend to be specularly reflective. Specular reflection refers to mirror-like reflection in which incident light is reflected at an angle that is equal to, but opposite that of the incident light. For example, if a beam of light hits a multilayer optical film at an angle of 45 degrees from the right, the light will be reflected at an angle of 45 degrees off to the left.

Reflective optical films of the present invention may also be in the form of diffusively reflective films comprising a first continuous phase having a second phase dispersed in the first phase, at least one of the phases being birefringent. Preferably, the dispersed phase comprises polymeric particles, and the diffusely reflective film is oriented, typically by stretching, in one or more directions. These films are described in U.S. Pat. Nos. 5,825,543, 5,867,316, and 5,783,120, the teachings of which are incorporated herein by reference. Diffusely reflective films useful in the invention may comprise one or more layers of poly(ethylene naphthalate) (PEN) or a copolymer of naphthalic acid, terephathalic acid, and ethylene glycol (CoPEN). The dispersed phase preferably comprises syndiotactic polystyrene, and may be incorporated into at least one of the layers. The characteristics of the dispersed phase (e.g., size, orientation, refractive index) can be manipulated to provide a range of reflective and transmissive properties to the film. In contrast to specular reflection associated with multilayer optical films, the diffuse reflection provides reflective luminance over a wide range of angles.

According to one approach, release coatings and/or heat sealable coatings are easily formed by coating a formulation comprising the desired polymer or blend of polymers onto the film from a suitable solvent or dispersion using conventional coating techniques such as gravure coating. The coating can then be dried and/or cured, as appropriate. According to another approach, a formulation comprising radiation curable monomer or oligomer components can be coated onto the film and then cured with a suitable source of radiation, e.g., ultraviolet radiation or e-beam irradiation. The use of such radiation curable materials is desirable because such materials can be coated at 100% solids without use of solvent.

Some embodiments of reflective optical films can be fabricated so as to be sufficiently brittle for use as a breakable liner. When a controlled depth cut is scored into such a film, the liner can easily be broken cleanly along the score when bent beyond the breaking point. Films with this kind of capability are desirable for certain kinds of packaging for patch therapeutic agent delivery devices.

Reflective optical films are well known in the optical film art, and many varieties of such films and corresponding methods of making those films have been described. Representative descriptions of reflective optical films is provided in U.S. Pat. Nos. 5,759,467; 5,882,774; 5,540,878; 5,448,404; 5,380,479; 5,234,729; 5,217,794; and 5,202,074. International patent publication WO 97/01440 also describes representative embodiments of reflective optical films, as do assignee's copending U.S. patent applications Ser. No. 09/006,288 filed Jan. 13, 1998; 09/229,724 filed Jan. 13, 1999; 09/006,591 filed Jan. 13, 1998; 09/006,086 filed Jan. 13, 1998; 09/126,917 filed Jul. 31, 1998; and 09/127,314 filed Jul. 31, 1998.

Multilayer optical films are also commercially available. For instance, such films are commercially available from the Minnesota Mining and Manufacturing Company (3M), St. Paul, Minn., as 3M Multilayered Colored Mirror Film. Such films may also be obtained under the trade designation “AURORA” special effect film from Engelhard Corp., Iselin, N.J. (also formerly available from the Mearl Corporation Peekskil, N.Y., under the trade designation “Mearl Iridescent Film”).

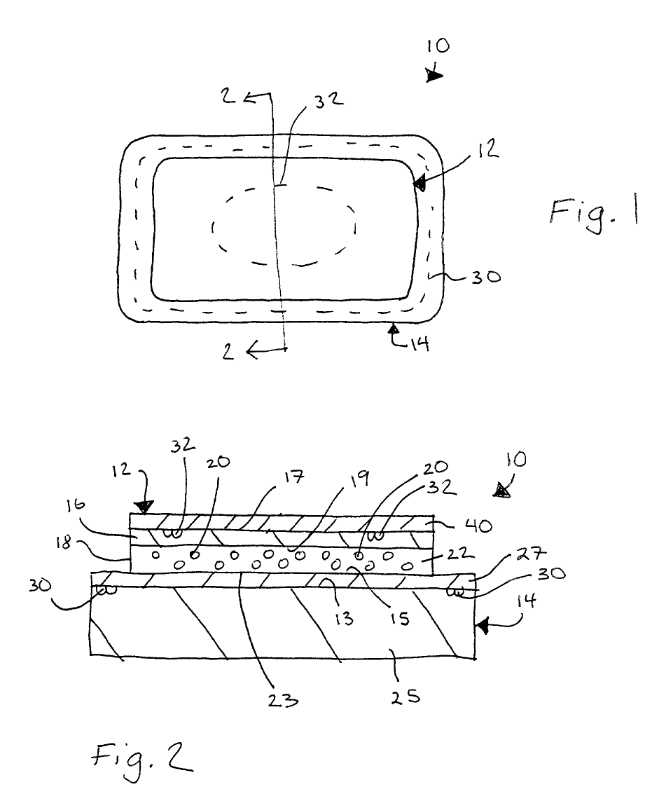

The principles of the present invention can be incorporated into a wide variety of different patch therapeutic agent delivery devices designed to be attached to a host for topical, transdermal, transmucosal, or other transtissue delivery of one or more therapeutically active agents. As one representative embodiment of such a structure, the present invention will now be described in connection with the assembly 10 illustrated in FIGS. 1 and 2. Assembly 10 generally includes patch device 12 releasably stored on release liner 14. Release liner 14 is formed from reflective optical film 25 and release coating 27 to provide release liner 14 with release surface 15. Patch device 12 generally includes backing 16 overlying and protecting repository layer 18. Backing 16, incorporating a reflective optical film, includes outer major surface 17 and inner major surface 19. Optional heat seal layer 40 is provided over backing 16.

Repository layer 18 comprises a dosage of therapeutically active agent 20 stored in matrix 22. Matrix 22 serves as a repository for storing therapeutically active agent 20. As illustrated, matrix 22 is a solid comprising a pressure sensitive adhesive so that surface 23 is tacky and can be used to attach patch device 12 to a host. Of course, the drug repository need not be a solid matrix comprising a pressure sensitive adhesive, but may also comprise another solid formulation, a gel, a semi-solid matrix, a fluid reservoir, and the like. Examples of other kinds of repository compositions and structures are described in U.S. Pat. Nos. 4,814,173; 4,834,979; 4,820,525; and 5,310,559.

A wide variety of pressure sensitive adhesives may be used to form matrix 22 of repository layer 18. Desirably, the particular pressure sensitive adhesive material to be used is a solid or semi-solid and is at least substantially chemically inert with respect to the other components of patch device 12, particularly the therapeutically active agent 20, or prodrug form thereof, if any. For therapeutic applications, the pressure sensitive adhesive material also should adhere well to the desired treatment site of the host. Preferably, the pressure sensitive adhesive material is water resistant so that patch device 12 remains adhered to the host for the desired treatment period even when exposed to moisture, but should be releasable so that patch device 12 can be removed when desired or when the expected delivery lifetime of therapeutically active agent 20 has been exceeded. The pressure sensitive adhesive material should also be compatible with the host so that undue irritation at the treatment site is avoided. Furthermore, the pressure sensitive adhesive material preferably is sufficiently flexible to allow patch device 12 to conform to and follow the contours of the treatment site without cracking and without causing undue restriction of host movement.

A particularly beneficial class of pressure sensitive adhesive materials meeting these criteria is the class of (meth)acrylate polymers, and in particular, the acrylate embodiments thereof. Representative embodiments of such (meth)acrylate pressure sensitive adhesives are described in U.S. Pat. Nos. 4,751,087; 4,737,577; 4,737,559; 4,693,776; and Re 24,906; and in WO 96/08229; the entire disclosures of each of which are incorporated herein by reference for all purposes.

A (meth)acrylate pressure sensitive adhesive suitable in the practice of the present invention preferably has a weight average molecular weight that is high enough so that the polymer has good handling, performance, and mechanical properties. However, if the weight average molecular weight of the (meth)acrylate pressure sensitive adhesive is too high, fluid compositions incorporating such adhesive may have a viscosity that is too high. Viscosity properties are of concern during manufacture of patch device 12 when repository layer 18 is formed from a solution or dispersion of ingredients including the pressure sensitive adhesive materials. Accordingly, a preferred (meth)acrylate pressure sensitive adhesive generally has a weight average molecular weight in a range such that the adhesive has an inherent viscosity in the range from about 0.2 dL/g to about 2 dL/g, more preferably from about 0.4 dL/g to about 1.4 dL/g. Inherent viscosity may be determined by conventional means using a Canon-Fenske #50 viscometer in a water bath controlled at 27° C. to measure the flow of 10 mL of polymer solution.

A particularly preferred (meth)acrylate pressure sensitive adhesive is a copolymer derived from monomers comprising, based upon the total weight of the copolymer, about 40 to about 100, preferably about 50 to about 75, weight percent of one or more alkyl (meth)acrylate monomers (A monomers) and 0 to about 60, preferably about 25 to about 50, weight percent of one or more free radically copolymerizable monomers (B monomers). Optionally, other monomers may also be incorporated into the copolymers. Such other monomers, for example, may further include up to about 30 weight percent, preferably up to about 15 weight percent, of a copolymerizable macromonomer as described in PCT Publication No. WO 96/08229.

The A monomer preferably is selected from one or more alkyl (meth)acrylates containing 1 to about 10 carbon atoms in the alkyl group. Representative examples of the alkyl (meth)acrylate monomer include methyl (meth)acrylate, n-butyl (meth)acrylate, n-pentyl (meth)acrylate, n-hexyl (meth)acrylate, isoheptyl (meth)acrylate, cyclohexyl (meth)acrylate, n-nonyl (meth)acrylate, n-decyl (meth)acrylate, isohexyl (meth)acrylate, 2-ethyloctyl (meth)acrylate, isooctyl (meth)acrylate, isobornyl (meth)acrylate, and 2-ethylhexyl (meth)acrylate. Combinations of these can be used if desired. Preferably, the alkyl (meth)acrylate is selected from isooctyl (meth)acrylate, butyl methacrylate, 2-ethylhexyl (meth)acrylate, cyclohexyl methacrylate, isobornyl methacrylate, and methyl methacrylate.

The copolymerizable B monomer is generally one or more (meth)acrylate monomers having at least one functional group selected from the grouping consisting of carboxylic acid, carboxylic acid ester, hydroxyl, anhydride, epoxy, thiol, isocyanate, sulfonamide, urea, carbamate, carboxamide, amine, ammonium, oxy, oxo, nitro, nitrogen, sulfur, phosphate, phosponate, cyano, combinations of these, and the like. Representative examples of specific materials that can be used singly or in combination as the B monomer include (meth)acrylic acid, maleic acid, vinyl acetate, a hydroxyalkyl (meth)acrylate containing about 2 to about 4 carbon atoms in the hydroxyalkyl group, (meth)acrylamide, an alkyl substituted (meth)acrylamide having 1 to about 8 carbon atoms in the alkyl group, diacetone (meth)acrylamide, a dialkyl (meth)acrylamide independently having 1 or 2 carbon atoms in each alkyl group, N-vinyl-N-methyl acetamide, N-vinyl valerolactam, N-vinyl caprolactam, N-vinyl-2-pyrrolidone, glycidyl (meth)acrylate, alkoxy (meth)acrylate containing 1 to 4 carbon atoms in the alkoxy group, 2-ethoxyethyl (meth)acrylate, 2,2-ethoxyethoxyethyl (meth)acrylate, furfuryl (meth)acrylate, tetrahydrofurfuryl (meth)acrylate, propylene glycol mono(meth)acrylate, polyethylene glycol (meth)acrylate, polyethylene glycol methyl ether (meth)acrylate, polyethylene oxide methyl ether (meth)acrylate, di(lower)alkylaminopropyl (meth)acrylamide (wherein lower means the alkyl moiety has 1 to 4 carbon atoms), (meth)acrylonitrile, combinations of these, and the like. Preferably, the copolymerizable B monomer is selected from hydroxyethyl acrylate, hydroxyethyl methacrylate, acrylamide, glyceryl acrylate, N,N-dimethyl acrylamide, 2-ethoxyethyl acrylate, 2,2-ethoxyethoxyethyl acrylate, tetrahydrofurfuryl acrylate, vinyl acetate, and acrylic acid. Any of the aforementioned alkyl groups may be linear, branched or cyclic.

One particularly preferred (meth)acrylate pressure sensitive adhesive is a copolymer formed by copolymerizing about 60 to about 80, preferably about 75 weight percent of isooctyl (meth)acrylate (preferably the acrylate form); about 1 to about 10, preferably about 5 weight percent of (meth)acrylamide (preferably the acrylate form); and about 5 to about 30, preferably about 20 weight percent of vinyl acetate. This (meth)acrylate pressure sensitive adhesive demonstrates excellent adhesion to the skin of a human or other animal host, is flexible and water resistant, is soluble in therapeutically compatible solvents such as isopropyl alcohol, and is very compatible with many kinds of therapeutically active agents. Other preferred (meth)acrylate pressure sensitive adhesive polymers are formed from monomers according to formulations summarized in the following table:

| PSA | Parts by weight: | | | | | | |
|---|---|---|---|---|---|---|---|
| Sample | IOA | ACM | VOAc | DMACM | AA | HEA | NVP |
| 1 | 93 | 7 | — | — | — | — | — |
| 2 | 70 | — | — | 30 | — | — | — |
| 3 | 63 | — | 37 | — | — | — | — |
| 4 | 80 | — | — | — | 20 | — | — |
| 5 | 60 | — | — | — | — | 40 | — |
| 6 | 91 | — | — | — | — | — | 9 |
| 7 | 89 | — | — | — | — | 2 | 9 |

wherein IOA is isooctyl acrylate, ACM is acrylamide, VOAc is vinyl acetate, DMACM is N,N-dimethylacrylamide, AA is acrylic acid, BEA is 2-hydroxyethyl acrylate, and NVP is N-vinylpyrrolidone.

The particularly preferred (meth)acrylate pressure sensitive adhesive may be prepared by free-radical polymerization methods known in the art, including but not limited to bulk, solution, emulsion and suspension polymerization methods. For example, according to the solution polymerization method, copolymers suitable for use in the present invention are prepared by dissolving the desired monomers in an appropriate solvent, adding a chain-transfer agent, a free-radical polymerization initiator, and other additives known in the art, sealing the solution in an inert atmosphere such as nitrogen or argon, and then agitating the mixture at a temperature sufficient to activate the initiator.

Solvents useful in such polymerizations can vary according to solubility of the monomers and additives. Typical solvents include ketones such as acetone, methyl ethyl ketone, 3-pentanone, methyl isobutyl ketone, diisobutyl ketone, and cyclohexanone; alcohols such as methanol, ethanol, propanol, n-butanol, isopropanol, isobutanol, cyclohexanol and methyl cyclohexanol; esters such as ethyl acetate, butyl acetate, isobutyl acetate, isopropyl acetate, and the like; aromatic hydrocarbons such as benzene, toluene, xylenes, cresol, and the like; ethers such as diisopropyl ether, diisobutyl ether, tetrahydrofuran, tetrahydropyran, and dioxane; and aprotic solvents such as dimethylformamide, dimethylsulfoxide and the like, and mixtures thereof Chain transfer agents suitable for solution polymerization include but are not limited to alcohols, mercaptans, certain halogenated small molecules, and mixtures thereof. Preferably, the chain transfer agent is chosen from the group consisting of carbon tetrabromide, isooctylthioglycolate, mercaptosuccinic acid, mercaptopropane diol, dodecyl mercaptan, ethanol and carbon tetrachloride. Most preferably, the chain transfer agent is mercaptopropane diol.

Free-radical polymerization initiators suitable for solution polymerization include those that are soluble in the reaction solvent and that are thermally activated, including but not limited to azo compounds, peroxides, and mixtures thereof. Useful peroxide initiators include those chosen from the group consisting of benzoyl peroxide, lauroyl peroxide, di-t-butyl peroxide and the like, and mixtures thereof. Useful azo compound initiators include those chosen from the group consisting of 2,2′-azobis (2-methylbutyronitrile); 2,2′azobis (isobutyronitrile); and 2,2′-azobis (2,4-dimethylpentanenitrile).

The (meth)acrylate pressure sensitive adhesive polymers may also be prepared by emulsion polymerization methods. According to the emulsion polymerization method, polymers are prepared by forming an emulsion comprising the desired monomers, a chain-transfer agent and a water-soluble redox-type initiator system in an inert atmosphere such as nitrogen or argon, and then heating the emulsion carefully until a reaction exotherm occurs. The reaction mixture is stirred and cooled and the resulting polymer is collected. Optionally, an ionic or nonionic surfactant may be added to the reaction mixture. Oxidation—reduction (“Redox”) free-radical initiators may also optionally be added. Redox initiators include, but are not limited to, those chosen from the group consisting of tertiary amines with organic peroxides (exemplified by the N, N-diethylaniline—benzoyl peroxide pair); organic halides with transition metal complexes (exemplified by the carbon tetrachloride—molybdenum hexacarbonyl pair); inorganic oxidation—reduction systems (exemplified by the potassium persulfate—sodium metabisulfite pair); and organic—inorganic systems (exemplified by the 2-mercaptoethanol—Fe⁺³ pair). Inorganic redox initiators are preferred because of their ease of handling and useful reaction temperature range.

The particular therapeutically active agent 20 used is not critical, but rather will depend upon the end use of patch device 12. Representative examples of therapeutically active agents that may be suitable for use in patch device 12 of the present invention include the following (grouped by therapeutic class) as well as prodrugs thereof:

Antidiarrhoeals such as diphenoxylate, loperamide and hyoscyamine;

Antihypertensives such as hydralazine, minoxidil, captopril, enalapril, clonidine, prazosin, debrisoquine, diazoxide, guanethidine, methyldopa, reserpine, trimethaphan;

Calcium channel blockers such as diltiazem, felodipine, amlodipine, nitrendipine, nifedipine and verapamil;

Antiarrhyrthmics such as amiodarone, flecainide, diisopyramide, procainamide, mexiletene and quinidine;

Antiangina agents such as glyceryl trinitrate, erythrityl tetranitrate, pentaerythritol tetranitrate, mannitol hexanitrate, perhexilene, isosorbide dinitrate and nicorandil,

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate;

Cardiotonic glycosides such as digoxin and other cardiac glycosides and theophylline derivatives;

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine;

Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol;

Antimigraine preparations such as ergotamine, dihydroergotamine, methysergide, pizotifen and sumatriptan;

Anticoagulants and thrombolytic agents such as warfarin, dicoumarol, low molecular weight heparins such as enoxaparin, streptokinase and its active derivatives;

Hemostatic agents such as aprotinin, tranexamic acid and protamine;

Analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenoperidine, codeine dihydrocodeine; acetylsalicylic acid (aspirin), paracetamol, and phenazone;

Hypnotics and sedatives such as the barbiturates amylobarbitone, butobarbitone and pentobarbitone and other hypnotics and sedatives such as chloral hydrate, chlormethiazole, hydroxyzine and meprobamate;

Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam;

Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium;

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline;

CNS stimulants such as caffeine, 3-(2-aminobutyl) indole; and methylphenidate (Ritalin);

Anti-alzheimer's agents such as tacrine;

Anti-Parkinson's agents such as amantadine, benserazide, carbidopa, levodopa, benztropine, biperiden, benzhexol, procyclidine and dopamine-2 agonists such as S(−)-2-N-propyl-N-2-thienylethylamino)-5-hydroxytetralin (N-0923);

Anticonvulsants such as phenytoin, valproic acid, primidone, phenobarbitone, methylphenobarbitone and carbamazepine, ethosuximide, methsuximide, phensuximide, sulthiame and clonazepam;

Antiemetics and antinauseants such as the phenothiazines prochloperazine, thiethylperazine and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride;

Non-steroidal anti-inflammatory agents including their racemic mixtures or individual enantiomers where applicable, preferably which can be formulated in combination with dermal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate;

Antirheumatoid agents such as penicillamine, aurothioglucose, sodium aurothiomalate, methotrexate and auranofin;

Muscle relaxants such as baclofen, diazepam, cyclobenzaprine hydrochloride, dantrolene, methocarbamol, orphenadrine and quinine;

Agents used in gout and hyperuricaernia such as allopurinol, colchicine, probenecid and sulphinpyrazone;

Oestrogens such as oestradiol, oestriol, oestrone, ethinyloestradiol, mestranol, stilboestrol, dienoestrol, epioestriol, estropipate and zeranol;

Progesterone and other progestagens such as allyloestrenol, dydrogesterone, lynoestrenol, norgestrel, norethyndrel, norethisterone, norethisterone acetate, gestodene, levonorgestrel, medroxyprogesterone and megestrol;

Antiandrogens such as cyproterone acetate and danazol;

Antioestrogens such as tamoxifen and epitiostanol and the aromatase inhibitors, exemestane and 4-hydroxy-androstenedione and its derivatives;

Androgens and anabolic agents such as testosterone, methyltestosterone, clostebol acetate, drostanolone, furazabol, nandrolone, oxandrolone, stanozolol, trenbolone acetate, -dihydro-testosterone, 17-α-methyl-1 9-nortestosterone and fluoxymesterone;

5-α Reductase inhibitors such as finasteride, turosteride, LY-191704 and MK-306;

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide;

Further examples of steroidal antiinflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, flucetonide, fludrocortisone acetate, betamethasone benzoate, chloroprednisone acetate, clocortolone acetate, descinolone acetonide, desoximetasone, dichlorisone acetate, difluprednate, flumethasone pivalate, flunisolide acetate, fluperolone acetate, fluprednisolone valerate, paramethasone acetate, prednisolamate, prednival, triamcinolone hexacetonide, cortivazol, formocortal and nivazol;

Pituitary hormones and their active derivatives or analogs such as corticotrophin, thyrotropin, follicle stimulating hormone (FSH), luteinising hormone (LH) and gonadotrophin releasing hormone (GnRH);

Hypoglycemic agents such as insulin, chlorpropamide, glibenclamide, gliclazide, glipizide, tolazamide, tolbutamide and metformin;

Thyroid hormones such as calcitonin, thyroxine and liothyronine and antithyroid agents such as carbimazole and propylthiouracil;

Other miscellaneous hormone agents such as octreotide;

Pituitary inhibitors such as bromocriptine;

Ovulation inducers such as clomiphene;

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene;

Antidiuretics such as desmopressin, lypressin and vasopressin including their active derivatives or analogs;

Obstetric drugs including agents acting on the uterus such as ergometrine, oxytocin and gemeprost;

Prostaglandins such as alprostadil (PGE1), prostacyclin (PGI2), dinoprost (prostaglandin F2-alpha) and misoprostol;

Antimicrobials including the cephalosporins such as cephalexin, cefoxytin and cephalothin;

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;

Tetracyclines such as minocycline, chlortetracycline, tetracycline, demeclocycline, doxycycline, methacycline and oxytetracycline and other tetracycline-type antibiotics;

Aminoglycosides such as amikacin, gentamicin, kanamycin, neomycin, netilmicin and tobramycin;

Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc pyrithione and sodium pyrithione;

Quinolones such as nalidixic acid, cinoxacin, ciprofloxacin, enoxacin and norfloxacin;

Sulphonamides such as phthalysulphthiazole, sulfadoxine, sulphadiazine, sulphamethizole and sulphamethoxazole;

Sulphones such as dapsone;

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; and benzoylperoxide;

Antituberculosis drugs such as ethambutol, isoniazid, pyrazinamide, rifampicin and clofazimine;

Antimalarials such as primaquine, pyrimethamine, chloroquine, hydroxychloroquine, quinine, mefloquine and halofantrine;

Antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;

Anthelmintics such as mebendazole, thiabendazole, niclosamide, praziquantel, pyrantel embonate and diethylcarbamazine;

Cytotoxic agents such as plicamycin, cyclophosphamide, dacarbazine, fluorouracil and its prodrugs (described, for example, in International Journal of Pharmaceutics 111, 223-233 (1994)), methotrexate, procarbazine, 6-mercaptopurine and mucophenolic acid;

Anorectic and weight reducing agents including dexfenfluramine, fenfluramine, diethylpropion, mazindol and phentermine;

Agents used in hypercalcaemia such as calcitriol, dihydrotachysterol and their active derivatives or analogs;

Antitussives such as ethylmorphine, dextromethorphan and pholcodine;

Expectorants such as carbolcysteine, bromhexine, emetine, quanifesin, ipecacuanha and saponins;

Decongestants such as phenylephrine, phenylpropanolamine and pseudoephedrine;

Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives;

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine;

Local anaesthetics such as bupivacaine, amethocaine, lignocaine, lidocaine, cinchocaine, dibucaine, mepivacaine, prilocaine; etidocaine; and procaine;

Stratum comeum lipids, such as ceramides, cholesterol and free fatty acids, for improved skin barrier repair [Man, et al., J. Invest. Dermatol., 106(5), 1096, (1996)];

Neuromuscular blocking agents such as suxamethonium bromide, alcuronium dichloride, pancuronium bromide, atracurium besylate, gallamine, tubocurarine and vecuronium bromide;

Smoking cessation agents such as nicotine, bupropion and ibogaine;

Insecticides and other pesticides which are suitable for local or systemic application;

Dermatological agents, such as vitamins A, C, B₁, B₂, B₆, B₁₂ₐ and E, vitamin E acetate and vitamin E sorbate; salts and esters of such vitamins; and provitamin forms;

Allergens for desensitisation such as house, dust or mite allergens;

Homeopathic agents;

Nutritional agents, such as vitamins, essential amino acids and essential fats;

Keratolytics such as the alpha-hydroxy acids, glycollic acid and salicylic acid;

Anti-acne agents such as isotretinoin, tretinoin and benzoyl peroxide;

Anti-psoriasis agents such as etretinate, cyclosporin and calcipotriol;

Anti-itch agents such as capsaicin and its derivatives such as nonivamide [Tsai, et al., Drug. Dev. Ind. Pharm., 20(4), 719, 1994];

Anticholinergic agents, which are effective for the inhibition of axillary sweating and for the control of prickly heat; such as methatropine nitrate, propantheline bromide, scopolamine, methscopolamine bromide, and quaternary acyloxymethyl ammnonium salts (described, for example, by Bodor et al., J. Med. Chem. 23, 474 (1980) and also in United Kingdom Specification No. 2010270, published Jun. 27, 1979); and

Other therapeutically active small to medium-sized peptides and proteins, e.g., vasopressin and human growth hormone and enzymes.

The amount of the therapeutically active agent is not particularly restricted but rather, may be limited by practical concerns. For example, if too much therapeutically active agent 20 is present, the adhesive properties of pressure sensitive matrix 22 may be reduced such that the pressure sensitive adhesive matrix 22 is not tacky enough to be attached to a host at the desired administration site. Also, therapeutically active agent 20 may be administered at too high a dosage rate if too much therapeutically active agent 20 is present. On the other hand, if too little of the therapeutically active agent 20 is present, then the rate at which the therapeutically active agent 20 is administered to the host may be too low.

The amount of the particular therapeutically active agent 20 to be included in repository layer 18 can be selected in accordance with conventional practices. Such amount will be dependent upon the particular therapeutically active agent or combination of therapeutically active agents used, the intended therapy, the characteristics of the intended host(s), the desired duration of the treatment, the length of time that the particular device 12 can be worn, and the like. Bearing these general guidelines in mind, repository layer 18 may include but are not restricted to 0.01 weight percent to about 40 weight percent of one or more therapeutically active agents 20 based upon the total weight of repository layer 18.

Optionally, in addition to the therapeutically active agent 20 and matrix 22, repository layer 18 may include other optional ingredients. One example of a preferred optional ingredient that is advantageously incorporated into repository layer 18 is one or more penetration enhancers. A penetration enhancer is an agent that improves the transtissue penetration rate of therapeutically active agent 20 through or deposit within a tissue such as skin, a mucosal membrane, or other tissue, whether such transtissue drug delivery is intended for local or systemic delivery.

If a penetration enhancer is to be included in repository layer 18, it is preferably included in an amount sufficient to cause delivery to occur at the desired rate. The amount of a penetration enhancer required to achieve such an objective can be determined by one skilled in the art in accordance with conventional practices. In determining a suitable amount of penetration enhancer to be used, the skilled worker would give due consideration to factors such as the nature of the other ingredients of the therapeutic agent delivery device, the nature of the penetration enhancer, the age and weight of the host, the nature of the host surface to which patch device 12 will be applied, and the like. As general guidelines, preferred therapeutic agent delivery devices of the present invention include about 1 part by weight to about 50 parts by weight, preferably about 5 parts by weight to about 40 parts by weight, more preferably about 10 parts by weight to about 30 parts by weight of the penetration enhancer per 100 parts by weight of repository layer 18.

Representative examples of penetration enhancers include esters of the type described in PCT Publication WO 97/29735, laurocaprolactone and its derivatives such as 1-alkylazacycoheptan-2-ones as described in U.S. Pat. No. 5,196,410; oleic acid and its ester derivates such as methyl oleate, ethyl oleate, propyl oleate, isopropyl oleate, butyl oleate, vinyl oleate, and glyceryl monooleate; sorbitan esters such as sorbitan monolaurate and sorbitol monooleate; other fatty acid esters such as glyceryl monolaurate, isopropyl laurate, isopropyl myristate, isopropyl palmitate, diisopropyl adipate, propylene glycol monolaurate, and propylene glycol monooleate; long chain alkyl esters of 2-pyrrolidone, such as 1-lauryl, 1-hexyl, and 1-(2-ethylhexyl)esters of 2-pyrrolidone; a penetration enhancer of the type described in U.S. Pat. No. 5,082,866 such as dodecyl (N,N-dimethylamino) acetate and dodecyl (N, N-dimethylamino) propionate; a penetration enhancer as described in U.S. Pat. No. 4,861,764 such as 2-n-nonyl-1-3-dioxolane; combinations of these, and the like. A specific example of a combination penetration enhancer is one that includes 10 to 70 parts by weight of isopropyl myristate, about 1 to about 25 parts by weight of glyceryl monolaurate, and about 5 to about 70 parts by weight of ethyl oleate per 100 parts by weight of the penetration enhancer.

Suitable penetration enhancers may also include anionic surfactants (sodium lauryl sulfate), cationic surfactants (cetylpyridinium chloride), nonionic surfactants (polysorbate 80, polyoxyethylene 9-lauryl ether, glyceryl monolaurate), bile salts and related compounds (sodium glycocholate, sodium taurocholate, sodium tauro-24,25-dihydrofusidate), combinations of these, and the like. Such penetration enhancers are also listed in U.S. Pat. Nos. 5,688,520 and 5,908,637.

In addition to, or in place of, one or more penetration enhancers, repository layer 18 may also include other ingredients such as flavorings, flavor masking agents, water soluble or water swellable fibrous reinforcers, coloring agents, fragrances, odor masking agents, solubilizers, solvent, fillers, antistatic agents, plasticizers, antioxidants, combinations of these, and the like.

In the practice of the present invention, at least one of release liner 14 and backing 16 comprises a reflective optical film. For purposes of illustration, both release liner 14 and backing 16 shown in FIGS. 1 and 2 comprise a reflective optical film. Advantageously this allows visual effects to be easily incorporated into assembly 10. For instance, in terms of color, each of release liner 14 and backing 16 independently can display an overall color, mirror-like finish, iridescence, combinations of these, and the like. Additionally, visually discernible patterns can also be formed on release liner 14 and/or backing 16 by forming a corresponding pattern of three-dimensional, topographic features on surfaces 13 and 17. For example, topographic features 30 are embossed onto surface 13 of release liner 14. Similarly, a graphical pattern of topographic features 32 in the form of an oval outline is embossed onto outer surface 17 of backing 16.

Of course, it is not required that both release liner 14 and backing 16 include a reflective optical film. To the extent that a reflective optical film is incorporated into release liner 14, but not backing 16, backing 16 may then be formed from any conventional backing material. Preferably, backing 16 may be formed from any flexible material that protects repository layer 18 from the environment, resists bulk fluid flow, provides a barrier against loss of the therapeutically active agent 20, and is substantially inert with respect to the ingredients incorporated into the other components of patch device 12. Backing 16 may be formed from one or more conventional materials typically used as backings for tapes, bandages, wound dressings, other transtissue therapeutic agent delivery devices, and the like. In the case of a composition that contains a drug intended to be delivered across a membrane such as skin or a mucusal membrane and intended to have systemic action, the backing is prefereably substantially resistant to the migration of the drug therethrough. In the case of a composition that contains a drug intended to be delivered, e.g. to the oral cavity or the vaginal cavity and/or intended to have local action, the backing can be permeable to the agent to be delivered and can be permeable to saliva as well. With respect to embodiments of the invention in which therapeutically active agent 20 is stored in a fluid reservoir, it may be desirable that backing 16 is heat sealable to itself and to a variety of other polymeric materials at relatively low temperatures. Alternatively, optional heat sealable layer 40 may be used for this purpose.

Representative examples of suitable backing materials include polyethylene, polypropylene, ethylene-vinyl acetate copolymers, polyurethane, ethylene propylenediene copolymer, polyisobutylene, other polyolefins, polyamide, polyester, combinations of these, and the like. Non-woven materials such as polyesters, polyolefins, and polyamides can also be used. Also a layer of hydrophobic elastomer such as polyisobutylene can function as a backing. Preferred backing materials include an acrylate pressure-sensitive adhesive coated polyurethane film such as TEGADERM brand transparent dressing (commercially available from Minnesota Mining and Manufacturing Company (3M), St. Paul, Minn.). Specific embodiments of suitable backing materials are further described in U.S. Pat. No. 5,372,819, the entire disclosure of which is incorporated by reference herein for all purposes. In some embodiments, backing 16 may be texturized as described in Assignee's patent application filed co-currently herewith and having Attorney Docket No. 54951USA5A in the names of Godbey et al. for PATCH THERAPEUTIC AGENT DELIVERY DEVICE HAVING TEXTURIZED BACKING, incorporated herein by reference in its entirety.

To the extent that a reflective optical film is incorporated into backing 16, but not release liner 14, release liner 14 may be any conventional type of release liner known in the art. In representative embodiments, such release liners may be a polymeric, paper, or metal film which can be coated on a surface with a suitable release coating, although some polymer films inherently provide release capabilities without the need for such a coating.

The therapeutic agent delivery devices in accordance with the present invention can be prepared by general methods well known to those skilled in the art. Embodiments in which the drug repository is a pressure sensitive adhesive matrix generally can be prepared, for instance, using methods set forth in U.S. Pat. Nos. 5,688,523; 4,714,655; 5,059,189; and 5,264,224. Those embodiments specifically intended to be used for transmucosal delivery and including a matrix in which the adhesive is a mucoadhesive can be made by methods set forth in WO 90/06505. When the repository is a gel, representative manufacturing methods are set forth in U.S. Pat. No. 4,834,979 and EP 0,556,158. Devices including a fluid reservoir in which the therapeutically active agent is stored may be prepared by the representative methods set forth in U.S. Pat. No. 4,834,979 and EP 0,556,158.

Assembly 10 of the present invention is easily used. Specifically, release liner 14 is removed and patch device 12 is then applied directly to the area of the host to be treated where it will desirably release a therapeutically effective amount of the agent at a suitable rate to the affected area. For the administration of systemic therapeutically active agents, patch device 12 can be applied to any suitable area of the patient's skin. Patch device 12 can also be applied to a mucosal surface, such as the oral mucosa. For transtissue applications, patch device 12 may be replaced as desired, as is necessary to maintain a therapeutically effective blood level of the therapeutically active agent or as is convenient to the user. Patch device 12 exhibits substantially sustained release of the therapeutically active agent such that a therapeutically effective blood level of the drug can be achieved and/or maintained for an extended period of time. Patch device 12 can also be used to maintain a desired level of the therapeutically active agent in the vicinity of the area to which the therapeutic agent delivery device is applied if the treatment being effected is desirably local rather than systemic.

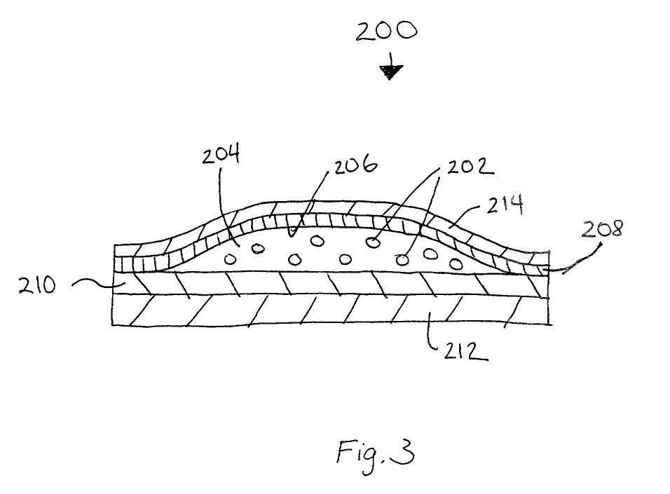

FIG. 3 shows an alternative embodiment of a patch 200 of the present invention in which therapeutically active agent 202 is releasably stored in liquid reservoir 204. Liquid reservoir 204, in turn, is stored in pocket 206 formed between heat seal layer 208 and permeable restraining membrane 210. Adhesive layer 212 serves to adhere patch 200 to an administration site. Barrier film 214 is provided over heat seal layer 208. Barrier film 214 advantageously incorporates a reflective optical film of the present invention.

The present invention will now be described in connection with the following illustrative examples.

### EXAMPLE 1

### Preparation of Comparison Samples

An adhesive formulation containing 10 percent by weight tetraglycol in a solvent-based acrylate copolymer adhesive was handspread coated 24 mils (610 micrometers) thick under laboratory cGMP conditions onto a 0.92 mil (23 micrometers), blue-dyed polyester film (Courtauld Performance Films, K021 Blue, CP Films Inc., Martinsville, Va.). The coated film was forced air dried at 125° F. (51.7° C.) for 10 minutes to remove residual solvent and to retain the tetraglycol. The sample was cut in half. One part (Sample A) was laminated to a 3 mil (76 micrometers), low density polyethylene film (3M CoTran #9720). The other part (Sample B) was laminated to a 3 mil (76 micrometers) fluoropolymer release-coated polyester film (3M Scotchpak #1022). Each sample was heatsealed into a pouch made from standard BAREX™/foil/polyester packaging film (Smurfit Flexible Packaging, Schaumburg, Ill.) and set aside for several days.

At the end of the dwell time, each pouch sample was opened and the adhesive was examined to see if any blue dye leached into the adhesive. The adhesive laminated to the polyester film was observed to have extracted a very noticeable amount of the blue dye. The adhesive laminated to the polyethylene film was slightly blue, and thus also extracted blue dye.

### EXAMPLE 2

### Preparation of Sample of the Invention

An adhesive formulation containing 10 percent by weight tetraglycol in a solvent-based acrylate copolymer adhesive was handspread coated 24 mils (610 micrometers) thick under laboratory cGMP conditions onto several yards of a 2 mil (51 micrometers) cyan/blue colored reflective optical film prepared as described in WO 99/36258, incorporated by reference. The surface of this film had been coated with the same fluoropolymer release coating as was used on the 3M Scotchpak™ #1022 film. The fluoropolymer release coating bearing the adhesive formulation was laminated to the 3M CoTran™ #9720 backing film. The resultant laminate structure was divided into several patch samples. These patch samples showed no color change over time. The adhesive layer, upon removal of the reflective optical film release liner, was still translucent, indicating that no dye or other colorant had leached into the adhesive by reason of there being no dye or colorant in the reflective optical film that could have been extracted by the adhesive.

### EXAMPLE 3

### Patch Rollstock Manufacture

A roll of 50 micrometer thick cyan/blue multilayer optical film was coated with a proprietary fluoropolymer release system similar to 3M Scotchpak #1022 Release Liner (3M, St. Paul, Minn.). This film was slit and used in a converting/die cutting process to manufacture placebo delivery patches in continuous roll form. Acrylate adhesive patches remained easy to remove from the release surface after one year in continuous contact. No color change or adhesion loss was noted.

### EXAMPLE 4

### Comparison of Heat Seal Properties

Samples of laminates containing a 13 μm layer of clear/cyan embossed multilayer optical film and 37 μm polyethylene/vinyl acetate copolymer (EVA) at 9%, 18% and 28% levels of vinyl acetate were heat sealed to matched samples of the same film (3M, St. Paul, Minn.). The clear/cyan film was prepared as described in Assignee's copending U.S. patent application Ser. No. 09/006086, Example 14. Embossing was carried out as described in Assignee's copending U.S. patent application Ser. No. 09/127065, Example 1. All samples were bonded (EVA sides together) at 163° C. for 10 seconds at 276 KPa with a 25.4 mm wide sealing bar installed on a Sentinel Heat-Sealer™ Model 12-AS (Packaging Industries Group Inc., Hyannis, Mass.). A control sample was also made using 3M Scotchpak™ 1012 heatsealable backing film (3M, St. Paul, Minn.) that used standard poly(ethylene terephthalate) film instead of the multilayer optical film samples. Triplicate samples of each were slit to 25.4 mm and tested for heat seal strength using an Instron™ Model 1122 (Instron Corp., Park Ridge, Ill.), running at 30.5 cm/min separation speed. Failure was deemed to occur if the seal failed or the film broke at 7.0 lbs force or less. All films met or exceeded this specification. No Engelhard/Mearl samples had a heat seal coating and, therefore, were not tested.

### EXAMPLE 5

### Suitability of Embossed Identification/Pattern for Skin Patch

Using a standard skin patch design, samples of a laminate including a 13 μm layer of clear/cyan embossed multilayer optical film and 37 μm polyethylene/vinyl acetate copolymer (EVA) at 9% were used to make placebo versions of active-in-adhesive skin patches. This was accomplished by adhering the multilayer optical film side of the film to #1524 Acrylate Transfer Adhesive (3M Medical Specialties, St. Paul, Minn.) on a silicone-coated paper release liner. This “sandwich” of materials was cut by hand using a steel rule die to a small oval size. The embossed pattern remained easily discernable through the EVA layer and was not effected by the adhesive. The multilayer optical film sample attached strongly to the adhesive layer and did not delaminate when removed after being worn on the forearm for eight hours.

Other multilayer optical films supplied by 3M and Engelhard may also be employed as a backing, bearing in mind that the Engelhard products, in particular, are thinner films that may be more difficult to handle, and the red and yellow dyes used on some versions are acetone soluble so formulations would need to be developed that minimize dye bleed.

### EXAMPLE 6

### Permanence of Embossed Identification/Pattern

Samples of a laminate including a 13 μm layer of clear/cyan embossed multilayer optical film and 37 μm polyethylene/vinyl acetate copolymer (EVA) at 9% were evaluated for pattern permanence. The embossed pattern could not be abraded (using 3M Scotchbrite™ Heavy-Duty Scouring Pad, 3M, St. Paul, Minn.) or eliminated from the patches by a solvent wipe with acetone. The surfaces were severely scratched by the scouring pad but no alteration of the embossed pattern was noticed. A piece of gravure-printed low density polyethylene that had been corona-treated before printing with a solvent-based ink was subjected to the same testing. The printing was easily abraded by the scouring pad and removed with the acetone wipe.

Other multilayer optical films supplied by 3M and Engelhard may also be embossed for permanent markings. The red and yellow dyes used on some versions of the Engelhard products may be solvated by the acetone wipe.

### EXAMPLE 7

### Moisture/Vapor and Oxygen Permeability

The permeability of several films to oxygen and to moisture/vapor was examined by industry-standard procedures.

Moisture/Vapor Transmission Rate (MVTR) was measured according to the method of ASTM F1249-90 using a Permatran™ W6 moisture/vapor transport device (Mocon, Minneapolis, Minn.) at 37.7° C. and 90% relative humidity on a 5 cm² sample of the film. Five readings were taken for each of three film samples, and the results were averaged and reported in Table 1.

Oxygen transportation rate was measured according to the method of ASTM D3585-95 using a Oxytran™ 1000H oxygen permeation device (Mocon, Minneapolis, Minn.) at 22.2° C. and 40% relative humidity for a sample size of 5 cm². All measurements were normalized for 760 mm Hg barometric pressure. Five readings were taken for each of two film samples, and the results were averaged and reported in Table 1.

TABLE 1

| | | MVTR | OTR |
|---|---|---|---|
| | Film | g/m²/day | cc/m²/day |
| | thickness, | per 25 μm | per 25 μm |
| Sample | μm | thickness | thickness |
| PET¹ Comparative | 53 | 22.32 | 144.91 |
| Cyan/Blue multilayer² | 45 | 18.74 | 72.32 |
| Magenta/Yellow multilayer² | 40 | 16.88 | 59.52 |
| Scotchpak ™ 1012, 9% EVA³ | 75 | 15.40 | 269.70 |
| Coated multilayer film⁴, 9% | 75 | 10.89 | 203.67 |
| EVA | | | |
| Coated multilayer film⁴, 18% | 78 | 10.45 | 149.92 |
| EVA | | | |
| Coated multilayer film⁴, 28% | 75 | 9.36 | 173.91 |
| EVA | | | |
| Mearl #8181 Blue/Violet⁵ | 23 | 88.47 | 114.39 |
| Mearl #8181 Red/Green⁵ | 23 | 76.28 | 230.18 |
| Mean #8601, Red/Red⁵ | 40 | 84.75 | 233.62 |
| Mean, Red⁵ | 30 | 70.02 | 273.42 |
| Mean, Yellow⁵ | 38 | 73.95 | 254.60 |

¹PET comparative film was poly(ethylene terephthalate) film prepared at 3M, St. Paul, MN.
²Cyan/blue and Magenta/yellow multilayer films were prepared as described in WO 99/36258, incorporated herein by reference.
³Scotchpak ™ 1012 film was a laminated poly(ethylene terephthalate)/ethylene vinyl acetate (9% vinyl acetate) backing film, commercially available from 3M, St. Paul, MN.
⁴Coated multilayer film was a multilayer optical film, approximately 12 microns thick, coated with approximately 63-65 microns of ethylene vinyl acetate copolymer, as described in Example 12 of copending U.S. Patent Application No. 09/006068, now allowed, the teachings of which are incorporated herein by reference.
⁵Mearl films were obtained from Engelhard Corp., Iselin, NJ. Where two colors are noted (i.e., “blue/violet”), the first color was the reflected color and the second was the transmitted color. Where one color is noted, it was the reflected color.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1. A therapeutic agent delivery device that can be applied onto a host in order to therapeutically administer a therapeutically active agent or prodrug form thereof to the host, said device incorporating at least one reflective optical film.

2. The therapeutic agent delivery device of claim 1, wherein the device comprises a backing, and wherein the reflective optical film forms at least a portion of the backing.

3. The therapeutic agent delivery device of claim 1, wherein the device comprises a release liner, and wherein the reflective optical film forms at least a portion of the release liner.

4. The therapeutic agent delivery device of claim 1, wherein the device comprises a release liner and a backing, wherein a first reflective optical film forms at least a portion of the release liner and a second reflective optical film forms at least a portion of the backing.

5. The therapeutic agent delivery device of claim 1, wherein the film comprises a plurality of topographic features that form a visually discernible pattern on the surface of the film.

6. The therapeutic agent delivery device of claim 1, wherein the film is a multilayer optical film.

7. The therapeutic agent delivery device of claim 1, wherein the film has a multiphase structure comprising a first birefringent phase and a second phase dispersed in the first phase.

8. The therapeutic agent delivery device of claim 1, wherein the film comprises an exterior surface and an interior surface and said pattern is formed on the exterior surface.

9. The therapeutic agent delivery device of claim 1, wherein the film comprises an exterior surface and an interior surface and said pattern is formed on the interior surface.

10. The therapeutic agent delivery device of claim 1, wherein the device is substantially free of any colorant, dye, ink, or toner.

11. A therapeutic agent delivery device that can be applied onto a host in order to therapeutically administer a therapeutically active agent or prodrug form thereof to the host, said device comprising:
(a) a repository comprising a releasably stored dosage of the therapeutically active agent; and
(b) a backing protecting the repository, wherein the backing comprises a first, reflective optical film.

12. The therapeutic agent delivery device of claim 11, wherein the device further comprises a release liner, and wherein a second, reflective optical film forms at least a portion of the release liner.

13. The therapeutic agent delivery device of claim 11, wherein the film comprises a plurality of topographic features that form a visually discernible pattern on the surface of the first film.

14. The therapeutic agent delivery device of claim 11, wherein the first film is a multilayer optical film.

15. The therapeutic agent delivery device of claim 11, wherein the first film has a multiphase structure comprising a first birefringent phase and a second phase dispersed in the first phase.

16. The therapeutic agent delivery device of claim 11, wherein the first film comprises an exterior surface and an interior surface and said pattern is formed on the exterior surface.

17. The therapeutic agent delivery device of claim 11, wherein the first film comprises an exterior surface and an interior surface and said pattern is formed on the interior surface.

18. The therapeutic agent delivery device of claim 11, wherein the device is substantially free of any colorant, dye, ink, or toner.

19. A therapeutic agent delivery device assembly, comprising:
(a) a therapeutic agent delivery device that can be applied onto a host in order to therapeutically administer a therapeutically active agent or prodrug form thereof to the host, said device comprising:
(i) a repository comprising a releasably stored dosage of the therapeutically active agent; and
(ii) a backing protecting the repository; and
(b) a release liner on which the therapeutic agent delivery device is releasably stored, said release liner incorporating a reflective optical film.

20. The therapeutic agent delivery device of claim 19, wherein the reflective optical film comprises a plurality of topographic features that form a visually discernible pattern on the surface of the film.

21. The therapeutic agent delivery device of claim 19, wherein the reflective optical film is a multilayer optical film.

22. The therapeutic agent delivery device of claim 19, wherein the reflective optical film has a multiphase structure comprising a first birefringent phase and a second phase dispersed in the first phase.

23. The therapeutic agent delivery device of claim 19, wherein the reflective optical film comprises an exterior surface and an interior surface and said pattern is formed on the exterior surface.

24. The therapeutic agent delivery device of claim 19, wherein the reflective optical film comprises an exterior surface and an interior surface and said pattern is formed on the interior surface.

25. The therapeutic agent delivery device of claim 19, wherein the device is substantially free of any colorant, dye, ink, or toner.

26. A method of making a therapeutic agent delivery device, comprising the steps of:
(a) forming a drug repository, said repository storing a dosage of a therapeutically active agent or prodrug form thereof, and
(b) causing the drug repository to be positioned in a therapeutic agent delivery device assembly, said assembly comprising a reflective optical film.

27. The method of claim 26, wherein the device comprises a backing, and wherein the reflective optical film forms at least a portion of the backing.

28. The method of claim 26, wherein the device comprises a release liner, and wherein the reflective optical film forms at least a portion of the release liner.

29. The method of claim 26, wherein the device comprises a release liner and a backing, wherein a first reflective optical film forms at least a portion of the release liner and a second reflective optical film forms at least a portion of the backing.

30. The method of claim 26, wherein the reflective optical film comprises a plurality of topographic features that form a visually discernible pattern on a surface of the film.

31. The method of claim 26, wherein the film is a multilayer optical film.

32. The method of claim 26, wherein the film has a multiphase structure comprising a first birefringent phase and a second phase dispersed in the first phase.

33. The method of claim 26, wherein the film comprises an exterior surface and an interior surface and said pattern is formed on the exterior surface.

34. The method of claim 26, wherein the film comprises an exterior surface and an interior surface and said pattern is formed on the interior surface.

35. The method of claim 26, wherein the device is substantially free of any colorant, dye, ink, or toner.

36. A method of therapeutically administering a therapeutically active agent or prodrug form thereof, comprising the steps of:
(a) providing an assembly comprising a patch therapeutic agent delivery device releasably supported upon a release liner, wherein the assembly comprises a reflective optical film and wherein the patch drug device comprises the therapeutically active agent releasably stored in a repository;
(b) removing the patch therapeutic agent delivery device from the release liner; and
(c) adhering the patch therapeutic agent delivery device to a host in a manner effective to cause the therapeutically active agent to be therapeutically administered to the host.

37. The method of claim 36, wherein the reflective optical film forms at least a portion of the backing.

38. The method of claim 36, wherein the device comprises a release liner, and wherein the reflective optical film forms at least a portion of the release liner.

39. The method of claim 36, wherein the device comprises a release liner and a backing, wherein a first reflective optical film forms at least a portion of the release liner and a second reflective optical film forms at least a portion of the backing.

40. The method of claim 36, wherein the film comprises a plurality of topographic features that form a visually discernible pattern on a surface of the film.

41. The method of claim 36, wherein the film is a multilayer optical film.

42. The method of claim 36, wherein the film has a multiphase structure comprising a first birefringent phase and a second phase dispersed in the first phase.

43. The method of claim 36, wherein the film comprises an exterior surface and an interior surface and said pattern is formed on the exterior surface.

44. The method of claim 36, wherein the film comprises an exterior surface and an interior surface and said pattern is formed on the interior surface.

45. The method of claim 36, wherein the device is substantially free of any colorant, dye, ink, or toner.
